(19) United States
(12) Patent Application Publication
(10) Pub. No.: US 2008/0015637 A1
(43) Pub. Date: Jan. 17, 2008

(54) Multi-nail hemorrhage occluder pin, hemorrhage occulder pin applicator

(76) Inventors: Shu Wen Jao, Taipei (TW); Wei Lee, Taipei (TW)

Correspondence Address: TROXELL LAW OFFICE PLLC, 5205 LEESBURG PIKE, SUITE 1404, FALLS CHURCH, VA, 22041, US

(21) Appl. No.: 11/484,644

(22) Filed: Jul. 12, 2006

(51) Int. Cl. A61B 17/08

(52) U.S. Cl. 606/219

(57) ABSTRACT

A multi-nail hemorrhage occluder pin is disclosed for effectively securing to the presacral bleeding defect to stop the bleeding during a proctectomy by means of the application of a metal hemorrhage occluder pin applicator. The multi-nail hemorrhage occluder pin has a head, and nails respectively formed of a series of barbed portions and perpendicularly extended from one side of the head for holding a bone or spongiahaemostic. The metal hemorrhage occluder pin applicator has a holder block with a recessed portion for receiving the multi-nail hemorrhage occluder pin for installation.

### BACKGROUND OF THE INVENTION

(a) Field of the Invention

The present invention relates to a surgical implement and more particularly, to a multi-nail hemorrhage occluder pin for fastening to the presacral bleeding area to stop the bleeding during a proctectomy.

(b) Description of the Prior Art

During a proctectomy, the sacral plexus at the pre-sacral area may be injured, causing a massive bleeding. In these cases, an electrocoagulation or suture process may be employed to stop the bleeding. However, these methods are not effective, and may result in an expansion of the bleeding area. Sterile gauze may also be used and inserted into the wound to stop the bleeding. However, when removing applied sterile gauze from the wound, the wound will soon start bleeding again. Bone wax may also be used for stopping a bleeding during a surgical treatment. However, bone wax may be washed away easily after having been applied to the sacral plexus. Recently, hemorrhage occluder pins are developed for application to the pre-sacral area to stop the bleeding. Conventional hemorrhage occluder pins simply have one single nail. These hemorrhage occluder pins are not practical for stopping a massive bleeding due to a large wound.

### SUMMARY OF THE INVENTION

According to one aspect of the present invention, a multi-nail hemorrhage occluder pin is to provide a more secure method to stop a presacral bleeding during a proctectomy by means of the application of a metal hemorrhage occluder pin applicator. The multi-nail hemorrhage occluder pin has a head, and 3 to 4 nails perpendicularly extended from one side of the head for holding a piece of bone or spongiahaemostic. The metal hemorrhage occluder pin applicator has a holder block with a recessed portion for receiving the multi-nail hemorrhage occluder pin for installation. According to another aspect of the present invention, the nails each are formed of a series of barbed portions to prevent backward displacement of the multi-nail hemorrhage occluder pin after installation.

### BRIEF DESCRIPTION OF THE DRAWINGS

FIG. 1 is an elevational view of a 4-nail type hemorrhage occluder pin according to the present invention.

FIG. 2 is an elevational view of a 3-nail type hemorrhage occluder pin according to the present invention.

FIG. 3 is a schematic exploded view showing the use of a hemorrhage occluder pin with a bone and a spongiahaemostic according to the present invention (I).

FIG. 4 is an assembly view of FIG. 3.

FIG. 5 is a schematic drawing showing the use of a hemorrhage occluder pin with a hemorrhage occluder pin applicator and a hammer according to the present invention.

FIG. 6 is a schematic drawing showing an application example of the hemorrhage occluder pin with the hemorrhage occluder pin applicator and the hammer in a proctectomy according to the present invention.

FIG. 7 is a schematic sectional view showing the hemorrhage occluder pin secured to the hemorrhage occluder pin applicator according to the present invention.

FIG. 8 is a schematic end view of the holder block of the hemorrhage occluder pin applicator according to the present invention.

### DETAILED DESCRIPTION OF THE PREFERRED EMBODIMENTS

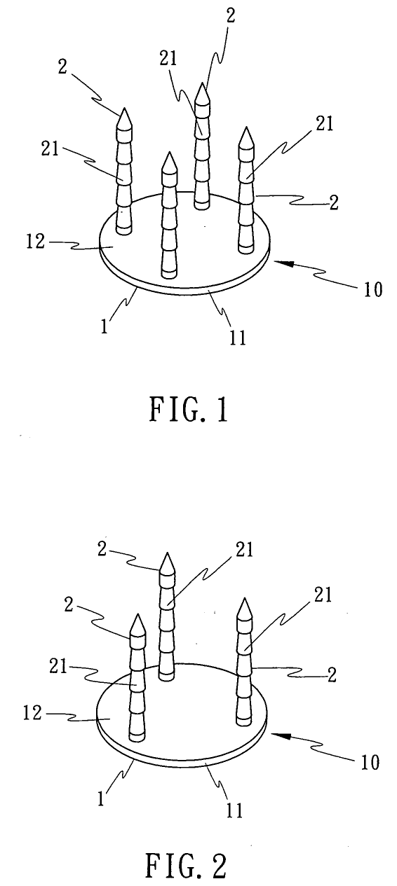

Referring to FIGS. 1 and 2, a multi-nail hemorrhage occluder pin 10 in accordance with the present invention is shown for use in a proctectomy. The multi-nail hemorrhage occluder pin 10 is made out of titanium alloy, comprising a head 1 and a plurality of nails 2. According to the embodiment shown in FIG. 1, the multi-nail hemorrhage occluder pin 10 has four nails 2. According to the embodiment shown in FIG. 2, the multi-nail hemorrhage occluder pin 10 has three nails 2.

The head 1 has an outer wall 11, and an inner wall 12 opposite to the outer wall 11. The outer wall 11 curves outwards. The inner wall 12 curves inwards. The size of the head 1 is determined subject to actual requirement.

The nails 2 are perpendicularly extended from the inner wall 12 of the head 1. The nails 2 may be equiangularly spaced from one another around the border area of the inner wall 12. Each nail 2 is comprised of a plurality of barbed portions 21 connected in series to prevent backward displacement after installation.

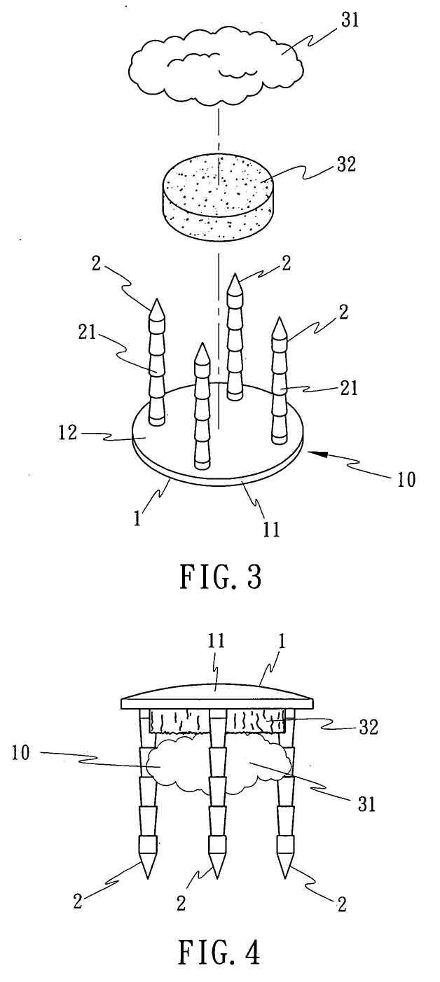

Referring to FIGS. 3 and 4, a piece of bone 32 (obtained from a bone bank) and a spongiahaemostic 31 may be set in between the nails 2 to stop the bleeding or to fill the presacral defect.

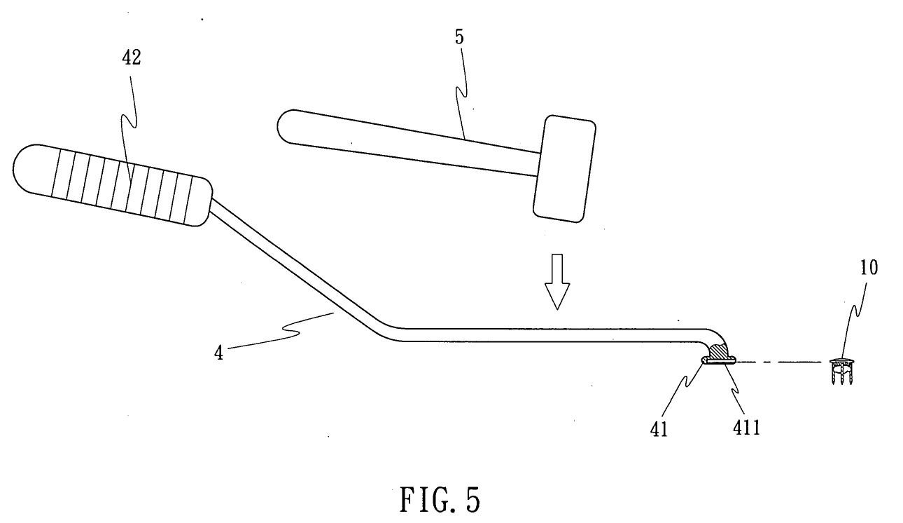

The multi-nail hemorrhage occluder pin 10 may be directly applied to the pre-sacral bleeding area with the fingers. Alternatively, a metal hemorrhage occluder pin applicator 4 and a surgical hammer 5 may be used to apply the multi-nail hemorrhage occluder pin 10 to the presacral bleeding area (see FIG. 5).

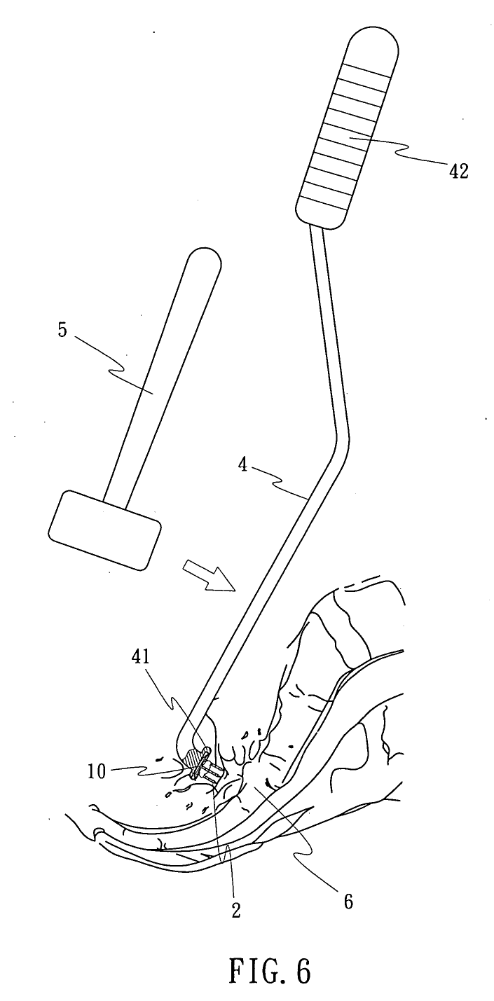

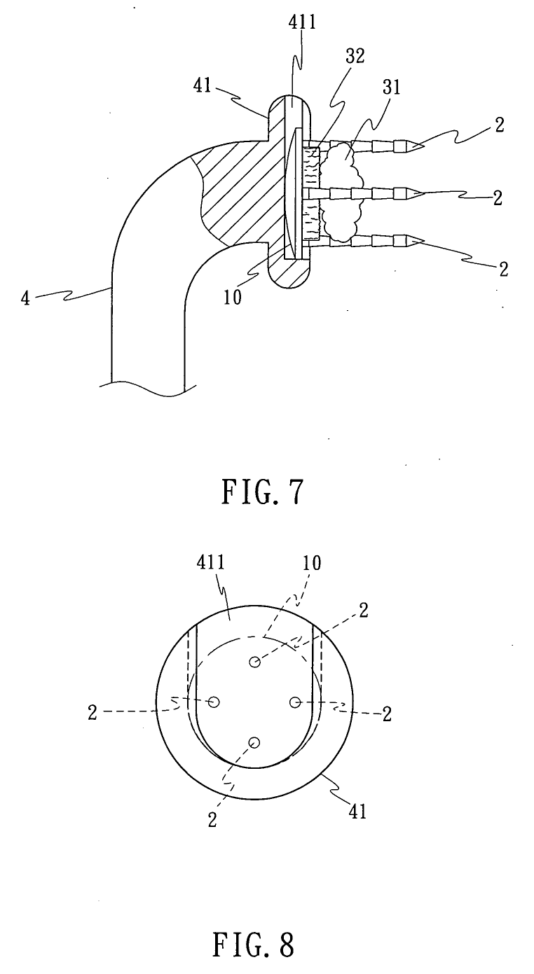

Referring to FIGS. 6˜8 and FIG. 5 again, the metal hemorrhage occluder pin applicator 4 has a holder block 41 at the front end, and a grip 42 at the rear end. The holder block 41 defines a recessed receiving portion 411 for receiving the head 1 of the multi-nail hemorrhage occluder pin 10. The metal hemorrhage occluder pin applicator 4 has a curved profile for convenient application. During application, the head 1 of the multi-nail hemorrhage occluder pin 10 is fitted into the recessed receiving portion 411 of the holder block 41 of the metal hemorrhage occluder pin applicator 4, and then the metal hemorrhage occluder pin applicator 4 is inserted into the body of the patient to hold the multi-nail hemorrhage occluder pin 10 on the pre-sacral bleeding area 6, and then the surgical hammer 5 is operated to strike the metal hemorrhage occluder pin applicator 4 and to further fasten the multi-nail hemorrhage occluder pin 10 to the pre-sacral bleeding area 6 to stop the bleeding. After installation of the multi-nail hemorrhage occluder pin 10, the metal hemorrhage occluder pin applicator 4 is removed from the multi-nail hemorrhage occluder pin 10.

Although particular embodiments of the invention have been described in detail for purposes of illustration, various modifications and enhancements may be made without departing from the spirit and scope of the invention. For example, the inner and outer walls of the head of the multi-nail hemorrhage occluder pin can be smooth walls. Accordingly, the invention is not to be limited except as by the appended claims.

What is claimed is:

1. A multi-nail hemorrhage occluder pin comprising:
a head, said head having an outer wall and an inner wall opposite to said outer wall; and
a plurality of nails perpendicularly extended from the inner wall of said head, said nails defining a space for holding a bone/spongiahaemostic.

2. The multi-nail hemorrhage occluder pin as claimed in claim 1, wherein said nails each are comprised of a plurality of barbed portions connected in series.

3. The multi-nail hemorrhage occluder pin as claimed in claim 1, wherein the outer wall of said head curves outwards.

4. The multi-nail hemorrhage occluder pin as claimed in claim 1, wherein the inner wall of said head curves inwards.

5. The multi-nail hemorrhage occluder pin as claimed in claim 1, wherein said nails are equiangularly spaced around the border of said inner wall of said head.

6. A metal hemorrhage occluder pin applicator for use with the multi-nail hemorrhage occluder pin as claimed in claim 1, comprising a curved profile, a grip at a first end thereof, and a holder block at a second end thereof, said holder block having a recessed receiving portion for receiving the head of the multi-nail hemorrhage occluder pin.

7. The metal hemorrhage occluder pin applicator as claimed in claim 1, which is made out of titanium alloy.
